(19) United States
(12) Patent Application Publication
(10) Pub. No.: US 2013/0065283 A1
(43) Pub. Date: Mar. 14, 2013

(54) PROCESS FOR OBTAINING FATTY ACID ALKYL ESTERS FROM LIPIDS IN A MEMBRANE CONTACTOR

(71) Applicants: Eddy G. Torp, Oslo (NO); Andreia Manuela Martins Miranda, Trondheim (NO); Inga Marie Aasen, Trondheim (NO)

(72) Inventors: Eddy G. Torp, Oslo (NO); Andreia Manuela Martins Miranda, Trondheim (NO); Inga Marie Aasen, Trondheim (NO)

(73) Assignee: DUE MILJØ AS, Oslo (NO)

(21) Appl. No.: 13/635,210

(22) PCT Filed: Mar. 16, 2011
(86) PCT No.: PCT/NO2011/000086

§ 371 Date: Nov. 28, 2012

(30) Foreign Application Priority Data
Mar. 17, 2010 (NO) 20100392

(51) Int. Cl. C12P 7/64

(52) U.S. Cl. 435/134

(57) ABSTRACT

The present invention relates to a new process for obtaining fatty acid alkyl esters from fatty acid containing lipids, i.e. mono-, di- and triglycerides and phospholipids, in a membrane contactor.

### 1. FIELD OF INVENTION

The present invention relates to a new process for obtaining fatty acid alkyl esters from fatty acid containing lipids, i.e. mono-, di- and triglycerides and phospholipids, in a membrane contactor.

### 2. BACKGROUND OF THE INVENTION

Long-chain omega-3 polyunsaturated fatty acids are essential fatty acids for humans and must be supplied by the diet. Of these, EPA (eicosapentaenoic acid, C20:5 Δ5,8,11,14,17) and DHA (docosahexaenoic acid, C22:6 Δ4,7,10,13,16,19) have particular health benefits, and are used not only as food supplements but also as pharmaceutical compounds for treatment of coronary diseases (Narayan et al., 2006, Food Rev Internat 22: 291-307). For enrichment of oils to be used in functional foods, animal feed and pharmaceutical applications, there is a need for concentrates of fatty acids or their alkyl ester derivatives.

Fatty acid alkyl esters are produced from vegetable and marine oils by reaction with an alcohol in the presence of a catalyst. Chemical transesterification of marine oils is carried out with a base catalyst or by e.g. sodium methoxide or sodium ethoxide, depending on the desired product. An alternative is enzymatic transesterification, or alcoholysis, by use of lipases. The chemical structure of the fatty acid and its position on the glycerol molecule affects the access of the enzyme. Therefore, the more easily accessible fatty acids will be first released. Enzymatic alcoholysis have been applied to enrich the glyceride fraction with LC-PUFA, such as EPA and DHA, as described by e.g. Haraldsson et al. (1997, JAOCS, 74: 1419-1424) and Lyberg and Adlercreutz (2008, Eur. J. Lipid Sci. Technol., 110: 317-324) and Patent No. US2006/0148047 A1.

The International Patent Application WO 2010/143974 A1 describes new processes for extracting fatty acids from aqueous biomass in a membrane contactor module. We have now found that a similar membrane contactor can also be used to fractionate fatty acid alkyl esters from lipids.

Membrane reactors have been used for isolation of fatty acid methyl esters (FAME) produced by chemical alcoholysis (WO 2006/089429 A1, CA2709575 A1, and WO 2009/077161 A2). These processes apply excess methanol and pressure as the driving force. In our new process, applying a membrane contactor, the concentration gradient created by the solubility of fatty acid alkyl esters in the different phases (partition coefficients) is the driving force. By combination of the membrane separation with an enzymatic alcoholysis with lipases that release the different alkyl esters sequentially, an enrichment of LC-PUFA alkyl esters is obtained.

### 3. DESCRIPTION OF THE INVENTION

Thus, one object of the present invention is to provide a new process for obtaining fatty acid alkyl esters (FAAE).

Another object of the present invention is to provide a new process for obtaining alkyl esters of long-chain polyunsaturated fatty acids (LC-PUFA).

Yet another object of the present invention is to provide a new process for obtaining alkyl esters of the omega-3 and/or omega- 6 fatty acids.

Yet another object of the present invention is to provide a new process for obtaining alkyl esters of the omega-3 fatty acids of DHA and EPA.

These and further objectives are achieved by the present invention.

The present invention relates to a process for fractionation of fatty acid alkyl esters (FAAE) from lipids in a membrane contactor, comprising the following steps:
a) enzymatic alcoholysis of the lipids to achieve a sequential release of FAAE
b) feeding the crude mixture of a) to the feed compartment A of said membrane contactor; and
c) separating the FAAE, as they are released, across the membrane to the product compartment B of said membrane contactor, containing an organic solvent or solvent mixture.

In this context, the term lipids include mono-, di- and triglycerides and phospholipids.

A schematic representation of the membrane contactor is set forth in FIG. 1.

is The enzymatic reaction takes place in an enzyme reactor R containing lipids, alcohol, enzymes and other solvents if necessary. The enzymes can be immobilized on an easily removable support. The reaction mixture, containing the released FAAE, is fed to the feed compartment A of the membrane contactor and back to the reactor R. An organic solvent or solvent mixture is circulated from a product recovery tank T to the product compartment B of the membrane contactor and back to the product recovery tank T, while the FAAE are transported across the membrane M by diffusion from compartment A to the compartment B where the FAAE accumulates. In FIG. 1, the arrow in the membrane indicates the direction of transport.

This new and inventive process utilizes enzymatic alcoholysis to achieve a sequential release of fatty acid alkyl esters, combined with membrane filtration for fractionation and separation of the FAAE. In the first stage, FAAE of saturated and monounsaturated medium to long chain fatty acids are formed, especially C16 and C18, by a first lipase that selectively attack the saturated and monounsaturated fatty acids due the structural and/or positional specificity. In the last stage, medium to long chain polyunsaturated fatty acid alkyl esters, especially DHA and EPA, can be achieved by leaving the first lipase to act for a longer period of time, or adding a different lipase. In between the first and the last stage, further stages may occur, depending on the fatty acid alkyl esters of interest. Most of the FAAE formed in the first stages are separated across the membrane before initiating the next stage.

Hydrophilic and hydrophobic membranes may be used, with hydrophobic membranes as the most likely choice if nonpolar solvents are applied.

Hydrophobic membrane may be made of any hydrophobic polymeric material, such as polyimides. Different polymers may be used, preferably, but not limited to Lenzing P84 and Matrimid 5218. Membranes may be reinforced by a porous supporting layer made of for instance non-woven polyester baking material.

Membranes applied in the present invention may be porous or nonporous membranes. Integrally skinned polyimide asymmetric membranes prepared by phase inversion may be applied (International Patent Application WO 2010/142979 A1. The membrane together with the distinct partition coefficients of each compound between the two phases creates a barrier which allows the separation of FAAE from the unreacted glycerides.

Membrane contactor module configuration is adapted in accordance with the membrane design chosen. Any of the designs known to those skilled in the art, such as tubular, hollow fibers or flat sheet membranes may be used in the present invention. The membrane can be configured with regard to any of the designs known, such as plate and frame, spiral wound, shell and tube, and derived designs thereof.

Lipid alcoholysis is carried out by reacting mono-, di-, and triglycerides and phospholipids with an alcohol, releasing FAAE. The reaction is catalyzed by one or more enzymes. The lipids may be of any origin, such as animal, vegetable or microbial, but of particular interest are marine and microbial oils that contain LC-PUFA, such as EPA and/or DHA. The marine oils may be from any marine biomass or animals, such as algae, zooplankton, fish and mammals.

The FAAE to be separated according to the present invention are any FAAE of interest. It might be alkyl esters of medium to long chain fatty acids comprising between fourteen and twenty carbons, either saturated or monounsaturated. Examples are the saturated fatty acids myristic acid (C14:0), palmitic acid (C16:0), stearic acid (C18:0) and arachidic acid (C20:0), and the monounsaturated oleic acid (C18:1) and gadoleic acid (C20:1). However, preferred FAAE to be separated according to the present invention are alkyl esters of long chain polyunsaturated fatty acids (LC-PUFA) with a is carbon chain longer than eighteen carbons and at least three double bonds, particularly EPA and DHA.

The enzymes applied are lipases, e.g., but not limited to, lipases of microbial origin, such as 1,3 position specific and non-specific lipases from Candida rugosa, Candida cylindracea, Candida antarctica, Pseudomonas sp, Mucor javanicus, Mucor mihei, Thermomyces lanuginosus (Lipozyme TL 100L), and mixtures thereof. Selectivity with respect to fatty acids and the releasing rates of the individual fatty acid alkyl esters are the criteria for the selection of the most suitable enzymes.

The lipases used according to the invention could be immobilized on an easily removable solid support. It is common practice to immobilize enzymes by adsorption for instance on celite particles (C. Torres et al, 2008, Biochemical Engineering Journal, 42: 105-110), polypropylene (A. M. Lyberg et al, 2008, Eur. J. Lipid Sci. Teclmol., 110: 317-324) or within the membrane (L. Giorno et al, 2006, Journal of Membrane Science, 276: 59-67) by entrapment. More recently support materials such as nanofibers and magnetic nanoparticles have been introduced (R. S. Prakasham et al, 2007, J. Phys. Chem. C., 111: 3842-3847; Z. G. Wang et al, 2009, J. Mol. Catal. B., 56: 189-195).

The alcohol employed in the alcoholysis reaction should preferably be selected from the lower alkyl alcohols (C1-C6), based on the application and/or demands of the further purification. Additional solvents in the feed phase may be considered if required to improve discrimination between reacted and unreacted glycerides and/or phase recirculation.

The product phase circulating from the product recovery tank T to the product compartment B of the membrane contactor and back to the product recovery tank T is initially filled with a suitable organic solvent or solvent mixture. Preferably, the solvent or solvent mixture is composed of an alcohol, as specified before, and/or a nonpolar solvent, preferably but not limited to hexane, cyclohexane, heptane, pentane, toluene, dichloroethane, dichloromethane, diethylether, ethylacetate, acetone, or any mixtures thereof.

In one embodiment, the stoichiometric amount of alcohol, or a small excess, and the immobilized enzyme are added to the lipids of the feed phase. Reaction is conducted in the enzyme reactor (R) until saturated and monounsaturated alkyl esters have been released and then fed to the compartment A of the membrane contactor. The feed phase is now composed of FAAE, unreacted glycerides and glycerol, and eventually a residual amount of alcohol. Only the released FAAE formed pass through the membrane to the product phase in compartment B, where receiving solvent or solvent mixture is circulating.

When the desired separation is achieved, an amount of alcohol necessary to react with the remaining mono and diglycerides is added in the enzyme reactor R. The first enzyme may now be replaced by another one. When alcoholysis is complete, feed phase is again fed to the compartment A of the membrane contactor and FAAE formed will pass through the membrane M to the product phase in compartment B, which has now been replaced by clean solvent or solvent mixture in use.

In between the first and the last stage, further stages may occur, depending on the fatty acid alkyl esters of interest.

The operating conditions will vary depending on the membrane, raw material, enzymes, solvent or solvent mixture and the fatty acid alkyl esters to be fractionated. Optimization of the operating conditions is within the general knowledge of the person skilled in the art, and will be made accordingly.

The process according to the invention may be operated as a batch process or more preferable, a semi-continuous or a continuous process. If a semi continuous or a continuous process is preferred, the concentration of ethanol in the reaction mixture is controlled by slow feeding, and the released FAAE is removed simultaneously by transport through the membrane.

In a preferred embodiment, palmitic (C16:0), stearic (C18:0) and oleic (C18:1) alkyl esters, and alkyl esters of other easily attacked fatty acids, are removed during first stage of the stepwise enzymatic alcoholysis. The alkyl esters of saturated and monounsaturated fatty acids constitute at least 50%, preferably at least 70%, most preferably at least 90% by weight of the total FAAE in the product phase separated in the first stage of the enzymatic alcoholysis. The fraction of DHA and EPA alkyl esters in the product phase at this stage should not be greater than 10%, more preferably not greater than 5%.

The main fraction of LC-PUFA alkyl esters is released in the last stage of the stepwise enzymatic alcoholysis, either by the continued action of the first enzyme or by a later added enzyme. In a preferred embodiment of the present invention the alkyl esters of long chain polyunsaturated fatty acids constitute at least 50%, preferably 60%, most preferably 80% by weight of the total fatty acid alkyl esters in the product phase separated in the last stage of the enzymatic alcoholysis. By selection of a suitable enzyme EPA may be separated from DHA in an intermediate step (Breivik et al., 1997, JAOCS, 74(11): 1425-1429).

After terminating the process according to the invention, the solvent or solvent mixture might be recovered. Solvent recovery is preferably achieved by organic solvent nanofiltration (OSN) rather than distillation in the proposed system. However, any suitable process for solvent mixture recovery might be used. The FAAE in the product recovery tank T is concentrated, while recovering the organic solvent by nanofiltration.

The concentrated FAAE obtained in the product recovery tank T may be further purified by methods well known by those skilled in the art, such as molecular distillation or chromatography, but particularly by high performance counter current chromatography (HPCCC).

### 4. FIGURES

FIG. 1 shows a schematic representation of the process according to the invention. The fatty acid alkyl esters rich phase (feed phase) is circulated from an enzyme reactor R to the compartment A of the membrane contactor and back to the enzyme reactor R. The organic solvent or solvent mixture is circulated from a product recovery tank T to the compartment B of the membrane contactor and back to the product recovery tank T. The fatty acid alkyl esters are transported across the membrane M by diffusion from the feed phase in the compartment A to the product phase in the compartment B where the product of interest accumulates.

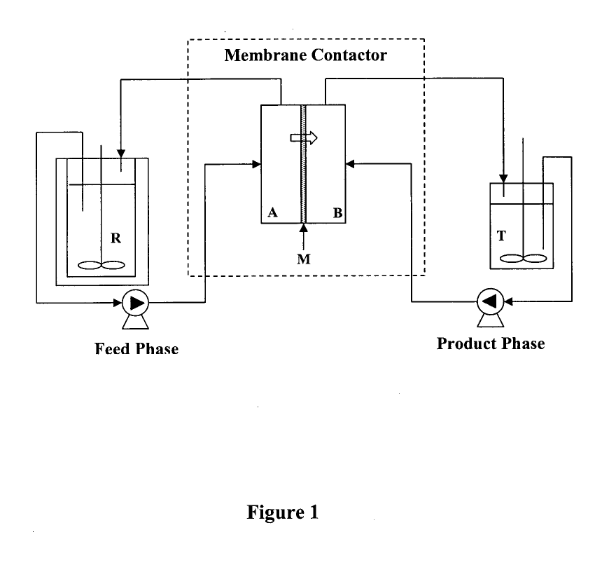

FIG. 2 shows the composition of the feed phase and accumulation of fatty acid ethyl esters (FAEE) in the product phase during the experiment described in Example 1. The FAEE was produced by chemical transesterification of cod liver oil with ethanol. All FAEE present in the feed phase had similar mass transfer rates across the membrane.

FIG. 3 shows concentrations of DHA and palmitic acid in the aqueous and organic phases throughout the experiment. Plots are divided in two sections. 1^{st} stage corresponds to the separation achieved when the first lipase was in use. As expected, concentration of C 16:0 in the organic phase was greater than the concentration of DHA at the end of this stage. 2^{nd} stage started when second lipase was added for the hydrolysis of remaining glycerides, consequently more DHA was released and concentration of DHA in the organic phase increased linearly.

### 5. EXAMPLES

The following examples illustrate the invention.

### Example 1

### Chemical Ethanolysis Followed by Membrane Separation

In order to demonstrate that fatty acid alkyl esters can be transported through a hydrophobic membrane, the following experiment was carried out:

A solution rich in fatty acid ethyl esters (FAEE) was prepared by chemical transesterification of cod liver oil with ethanol. 50 ml of ethanol (99.9%) containing 1.5% potassium hydroxide was added to 184 g of cod liver oil (Møller's tran, Axellus A S, Norway) in an Erlenmeyer flask. After being flushed with nitrogen and properly sealed, the flask was placed in a water bath at 55° C. and the mixture was stirred for 30 minutes at 1000 rpm. When chemical ethanolysis was complete, stirring was stopped and the mixture left standing for 30 minutes. After that time, a heavy phase (glycerol) and a light phase (ethyl esters) were perfectly formed and distinguished. 50 ml of the light phase, containing the fatty acid ethyl esters, were withdrawn to a clean Erlenmeyer flask and diluted five fold by addition of 200 ml of ethanol (99.9%). This solution was further used as the feed phase in the membrane separation test.

In the membrane contactor, an asymmetric polyimide membrane was used. Matrimid 5218 was chosen because of the well known hydrophobic characteristics of this polyimide. The flat sheet membrane was prepared by phase inversion; dope solution was prepared by dissolving the required amount of polymer in dimethylformamide (DMF). Membrane had a molecular weight cut off of ˜35 kDa and the filtration area in the membrane contactor was 41 cm².

The FAEE solution in ethanol (feed phase) and the organic solvent (product phase, in this case also ethanol), separated by the hydrophobic membrane, were circulating continuously (gear pump), one on each side of the membrane. The flow rate was the same at both sides, required to avoiding phase breakthrough, since the same solvent was used on both sides.

Initial volumes of feed and product phases were 250 and 200 ml, respectively. Experiment was conducted at room temperature and atmospheric pressure for 8 hours. Samples (1 ml) were withdrawn from both phases at 1, 2, 5 and 8 hours. After evaporation of ethanol under nitrogen, FAEE were dissolved in hexane containing 0.02% methyl heneicosanoate (>99% purity, internal standard) and 0.5% BHT. Samples were further analysed by GC for the quantification of FAEE.

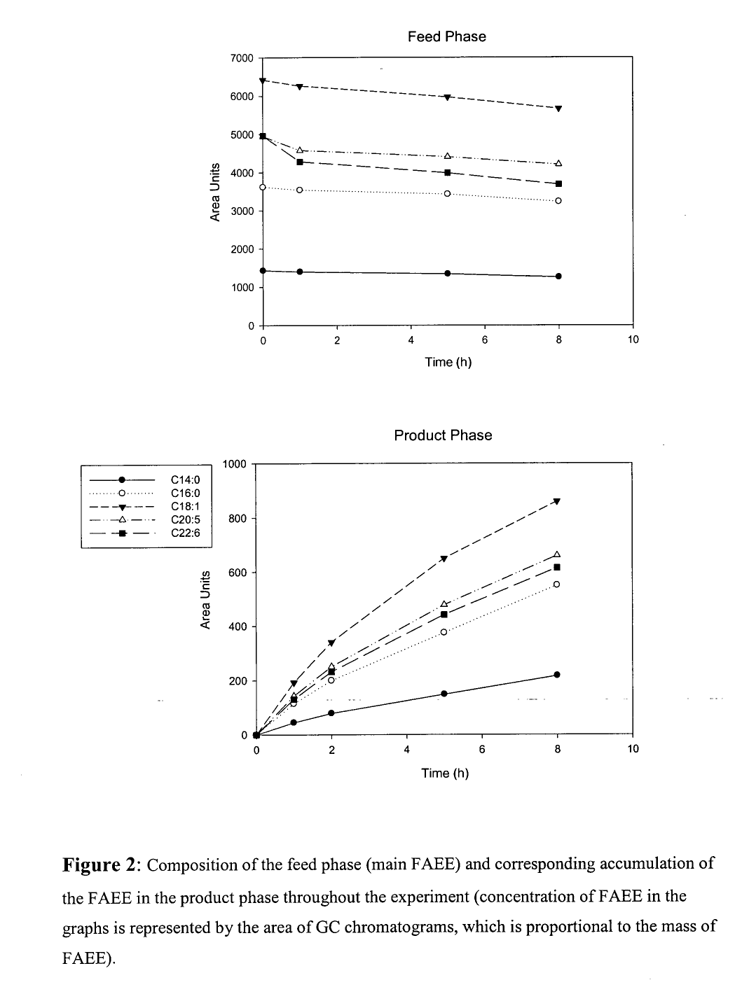

The composition of the feed phase (main FAEE) and the corresponding accumulation of the FAEE in the product phases throughout the experiment is depicted in FIG. 2. 14% of total FAEE in the initial feed phase were transferred to the product phase after 8 hours. All FAEE present in the feed phase had similar mass transfer rates across the zo membrane.

This example demonstrates that fatty acid alkyl esters can be transported from a fatty acid alkyl ester rich phase through a hydrophobic membrane to a product phase in a membrane contactor system.

### Example 2

### Fractionation of Fatty Acids by Sequential Enzymatic Hydrolysis and Simultaneous Fractionation in a Membrane Contactor

This example illustrates the principle of fractionation of saturated and polyunsaturated fatty acids by performing enzymatic lipid hydrolysis releasing saturated and/or monounsaturated fatty acids and PUFA sequentially, with simultaneous extraction of the released fatty acids through a membrane in a membrane contactor system.

150 ml of a suspension of triglyceride released from algal biomass in 0.1M potassium phosphate buffer (pH 7), containing 2 wt % lipids with fatty acid composition as given in Table 1 was used as raw material/feed solution. 100 ml of ethanol were added to the triglyceride suspension, the flask was incubated in a water bath at 37° C. and the first lipase was added. Recirculation of the triglyceride suspension to the aqueous compartment of the membrane contactor was initiated 2 hours after the beginning of the triglyceride hydrolysis. The second lipase was added to the biomass suspension when saturated fatty acids were satisfactorily removed and solvent in the organic phase was replaced by clean solvent.

TABLE 1
Fatty acid composition of triglyceride suspension, i.e. feed solution

| | Total amount |
|---|---|
| Fatty Acid (FA) | (mg FA in 250 ml TG suspension) |
| C16:0 | 675 |
| C18:1 | 315 |
| C22:6 | 875 |

Hydrophobic membrane used and the experimental set up was the same as in example 1. The two liquid circuits, separated by the hydrophobic membrane, namely the fatty acids rich phase (triglyceride suspension) and the organic phase, were in continuous circulation (gear pump), one on each side of the membrane contactor. The fatty acids rich phase was circulating at a higher flow rate (90 L/h) than that of the organic phase zo (20 L/h), in order to avoid water breakthrough and also to facilitate the membrane to be wetted by the solvent. Initial volumes of fatty acids rich phase and organic phase were 250 ml and 200 ml, respectively. Experiments were conducted at atmospheric pressure. Samples were collected periodically and immediately methylated to be further analysed by GC.

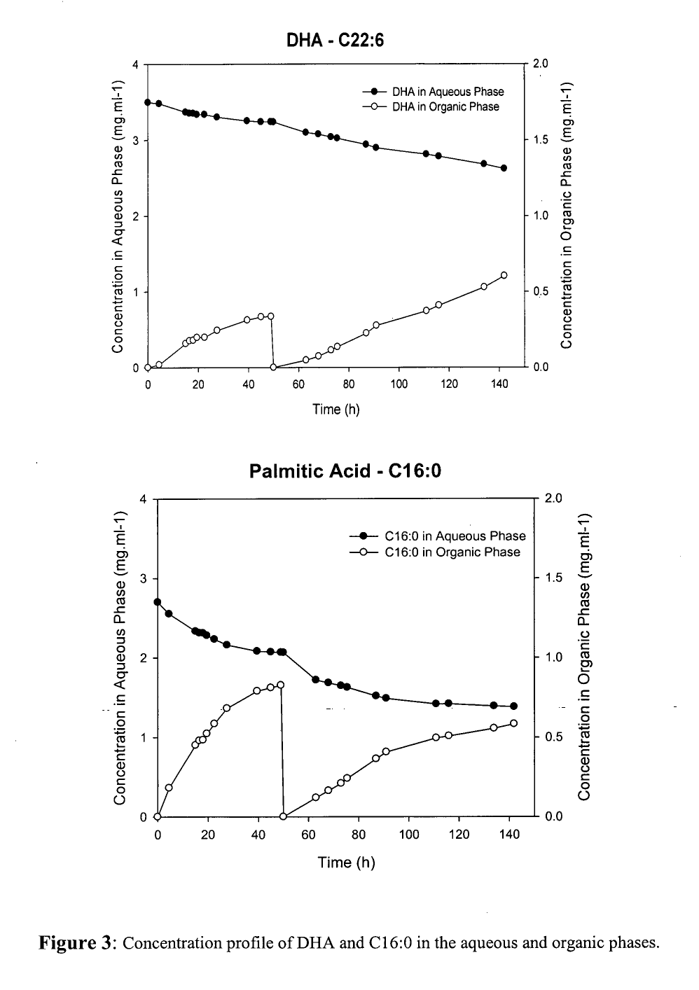

As depicted in FIG. 3, these experiments proved that saturated fatty acids, namely C 16:0, were preferably extracted in the first stage, while polyunsaturated fatty acids remain on the retentate side. By the addition of the second lipase, remaining glycerides were hydrolysed. Therefore, at the second stage of the experiment, an enhancement of the mass transfer rates of polyunsaturated fatty acids, namely DHA, was observed. By the end of this experiment, DHA transport was still increasing linearly, whilst C16:0 was stabilizing.

1. Process for fractionating fatty acid alkyl esters (FAAE) from lipids in a membrane contactor, comprising the following steps:
a) enzymatic alcoholysis of the lipids to achieve a sequential release of FAAE;
b) feeding the crude mixture of a) to the a feed compartment A of said membrane contactor; and
c) separating the FAAE, as they are released, across a membrane M to a product compartment B of said membrane contactor, containing an organic solvent or solvent mixture.

2. Process according to claim 1, wherein
a) the enzymatic reaction takes place in an enzyme reactor R containing lipids, alcohol, enzyme,
b) the reaction mixture containing the released FAAE, is fed to the feed compartment A of the membrane contactor and back to the reactor R,
c) an organic solvent or solvent mixture is circulated from a product recovery tank T to the product compartment B of the membrane contactor and back to the product recovery tank T, and
d) the FAAE crossing the membrane M by diffusion from compartment A to the product compartment B, thereby accumulating FAAE.

3. (canceled)

4. Process according to claim 1, wherein the enzymes are immobilized on a removable support.

5. Process according to claim 1, wherein FAAE are released sequentially, the saturated and monounsaturated medium to long chain fatty acids being substantially released in a first stage and the polyunsaturated fatty acids being substantially released in a last stage.

6. Process according to claim 5, wherein the alkyl esters of the saturated and monounsaturated fatty acids constitute at least 50%, by weight of the total FAAE in the product phase separated in the first stage.

7. Process according to claim 5, wherein the alkyl esters of the long chain polyunsaturated fatty acids constitute at least 50%, preferably at least 60%, most preferably at by weight of the total FAAE in the product phase separated in the last stage.

8. Process according to claim 1, wherein lipids are selected from the consisting of mono-, di- and triglycerides and phospholipids of any origin.

9. Process according to claim 1, wherein the alcohol is selected from the group of lower alkyl alcohols (C1-C6).

10. Process according to claim 1, wherein said solvent or solvent mixture is selected from the group consisting of lower alkyl alcohols and the group of nonpolar solvents, and mixtures thereof.

11. Process according to claim 6, wherein the alkyl esters of the saturated and monounsaturated fatty acids constitute at least 70% by weight of the total FAAE in the product phase separated in the first stage.

12. Process according to claim 11, wherein the alkyl esters of the saturated and monounsaturated fatty acids constitute at least 90% by weight of the total FAAE in the product phase separated in the first stage.

13. Process according to claim 7, wherein the alkyl esters of the long chain polyunsaturated fatty acids constitute at least 70% by weight of the total FAAE in the product phase separated in the last stage.

14. Process according to claim 13, wherein the alkyl esters of the long chain polyunsaturated fatty acids constitute at least 90% by weight of the total FAAE in the product phase separated in the last stage.
